(19) United States
(12) Patent Application Publication
(10) Pub. No.: US 2016/0058311 A1
(43) Pub. Date: Mar. 3, 2016

(54) BIOLOGICAL INFORMATION DETECTING DEVICE

(71) Applicant: Seiko Epson Corporation, Shinjuku-ku (JP)

(72) Inventors: Atsushi KONDO, Azumino-shi (JP); Yoshihiro Hidai, Shiojiri-shi (JP)

(21) Appl. No.: 14/834,229

(22) Filed: Aug. 24, 2015

(30) Foreign Application Priority Data
Aug. 27, 2014 (JP) 2014-172651

(51) Int. Cl. A61B 5/024; A61B 5/00

(52) U.S. Cl. CPC A61B 5/02427; A61B 5/681; A61B 5/6843; A61B 5/7455; A61B 5/721; A61B 2562/0233; A61B 2562/185; A61B 2562/12; A61B 2560/0214; A61B 2562/16

(57) ABSTRACT

A biological information detecting device includes a sensor detecting biological information of a subject, and a case housing the sensor, the case includes a light transmitting section formed of a resin material, the light transmitting section including a detection window through which light incident on the sensor is transmitted, and a light shielding section formed of a glass-containing resin material, the light shielding section disposed in the vicinity of the light transmitting section, wherein the light transmitting section and the light shielding section are integrally formed.

This application claims priority to Japanese Patent Application No. 2014-172651, filed Aug. 27, 2014, the entirety of which is hereby incorporated by reference.

### BACKGROUND

1. Technical Field

The disclosure relates to a biological information detecting device.

2. Related Art

Recently, a wristwatch type activity meter has been widely used according to the improvement of a sensing technology. As a sensor used in the activity meter, an acceleration sensor, a gyro sensor, a Global Positioning System (GPS), and the like are considered. By using such a sensor, information such as a motion, a position, a movement distance, and a movement route of a body of a user is able to be acquired.

In addition, a recent activity meter may sense not only the activity of the user which is known from the outside as described above, but also inner biological activity. For example, it may be proposed that a pulse wave sensor is disposed in the activity meter, and pulse wave information (a pulse rate, a pulse interval, and the like) of the user is acquired by using the pulse wave sensor.

When such a device is intended to be realized, the structure of the device is set to be suitable, and thus it is possible to perform sensing with high accuracy, to increase the comfort of the user, or to increase aesthetic appearance.

For example, in JP-A-2012-90975, a structure of a band which is preferable for a wristwatch type device or the like is disclosed.

When a photoelectric sensor is used as the pulse wave sensor, reflected light reflected on a subject (in particular, a portion in which a blood vessel of a measurement target is included) includes a pulse wave component, and thus has to be received, but the other light becomes a noise component, and thus has to be shielded. For this reason, a biological information detecting device may be configured to include a light transmitting section and a light shielding section in order to transmit necessary light and to shield unnecessary light.

In this case, the light transmitting section and the light shielding section may be realized by a simple method, and in order to protect a circuit board or the like which is disposed in a device, the necessity of waterproof properties increases, and in order to reduce a mounting burden of a user in a wristwatch type wearable device, the necessity of a reduction in size (thickness) and weight also increases. However, in a method of JP-A-2012-90975 or the like, a structure of a biological information detecting device satisfying these various demands is not disclosed.

### SUMMARY

An advantage of some aspects of the disclosure is to provide a biological information detecting device or the like.

An aspect of the disclosure relates to a biological information detecting device including a sensor detecting biological information of a subject; and a case housing the sensor, in which the case includes a light transmitting section including a detection window through which light incident on the sensor is transmitted, and a light shielding section arranged in the vicinity of the light transmitting section, the light transmitting section and the light shielding section are integrally formed, the light transmitting section is formed of a resin material, and the light shielding section is formed of a glass-containing resin material in which glass is contained in a resin material.

In the aspect of the disclosure, in the biological information detecting device including the light transmitting section and the light shielding section for receiving and shielding the light with respect to the sensor, the light shielding section is formed of the glass-containing resin material. Accordingly, the strength of the light shielding section configuring the case is able to increase, and thus it is possible to suppress inflow of moisture or the like due to a modification or the like or damage in internal parts. Further, at the time of increasing the strength, it is not necessary to increase the thickness of the member, and thus it is possible to reduce the thickness and the weight of the biological information detecting device.

In the biological information detecting device according to the aspect of the disclosure, the light transmitting section and the light shielding section may be integrally formed by two-color molding or insert molding.

With this configuration, it is possible to easily form the light transmitting section and the light shielding section.

In the biological information detecting device according to the aspect of the disclosure, the light shielding section may be arranged to be superimposed on the light transmitting section from the subject side of the light transmitting section in a portion other than the detection window.

With this configuration, it is possible to prevent the light from being incident on the biological information detecting device (in a narrow sense, the sensor) in the portion other than the detection window.

In the biological information detecting device according to the aspect of the disclosure, the case may include a top case, and a bottom case including the light transmitting section and the light shielding section, the light transmitting section may extend from the detection window to a sealing portion which is disposed in a connection portion between the top case and the bottom case.

With this configuration, it is possible to realize waterproofness between the light transmitting section and the light shielding section by using the sealing portion disposed between the top case and the bottom case.

In the biological information detecting device according to the aspect of the disclosure, the sealing portion may include a packing which seals the case.

With this configuration, it is possible to realize the waterproofness of the sealing portion by using the packing.

In the biological information detecting device according to the aspect of the disclosure, the light transmitting section may be arranged in a region between the detection window and the sealing portion in a plan view in which the bottom case is seen from a direction perpendicular to a contact surface with respect to the subject.

With this configuration, it is possible to dispose the light transmitting section in a suitable region in a relationship between the detection window and the sealing portion.

In the biological information detecting device according to the aspect of the disclosure, the light transmitting section may contain at least one of a polycarbonate, an ABS resin, and an acrylic resin, and the light shielding section may contain at least one of the polycarbonate containing glass, the ABS resin containing glass, and the acrylic resin containing glass.

With this configuration, it is possible to use various resins as the resin material of the light transmitting section and a target of the light shielding section in which glass is contained.

In the biological information detecting device according to the aspect of the disclosure, the light transmitting section may further include a convex portion applying a pressing force to a biological surface of the subject by being in contact with the biological surface at the time of being mounted on the subject, and a groove portion disposed in the vicinity of the convex portion.

With this configuration, it is possible to efficiently apply the pressing force to the subject by using the convex portion.

The biological information detecting device according to the aspect of the disclosure may further include a pressing force suppressor which is arranged in the vicinity of the convex portion in a plan view in which the case is seen from a direction perpendicular to a contact surface with respect to the subject, and suppresses the pressing force applied to the subject by the convex portion.

With this configuration, it is possible to stabilize the pressing force of the convex portion with respect to the subject.

In the biological information detecting device according to the aspect of the disclosure, the sensor may include a first light receiver and a second light receiver which receive light from the subject.

With this configuration, it is possible to dispose a plurality of light receivers in a photoelectric sensor.

In the biological information detecting device according to the aspect of the disclosure, in a direction from the sensor to the subject, when a height of a position or a region of the light transmitting section corresponding to the first light receiver is h1, and a height of a position or a region of the light transmitting section corresponding to the second light receiver is h2, h1>h2 may be satisfied.

With this configuration, it is possible to change the height of the light transmitting section (in a narrow sense, the convex portion) by the first light receiver and the second light receiver, and it is possible to output a signal having different properties in each of the light receivers.

The biological information detecting device according to the aspect of the disclosure may further include a secondary battery housed in the case; and a circuit board electrically connected to the sensor, and the secondary battery may be arranged between the circuit board and the sensor.

With this configuration, it is possible to perform various types of processing (controls) by the biological information detecting device, and it is possible to supply power used for executing the processing or driving other parts from a battery in the device.

In the biological information detecting device according to the aspect of the disclosure, a vibrator may be arranged between the secondary battery and the case in a plan view in which the case is seen from a direction perpendicular to a contact surface with respect to the subject.

With this configuration, it is possible to dispose the vibrator which is able to be used in a user interface or the like, and it is possible to efficiently arrange the vibrator and the secondary battery in the biological information detecting device.

Another aspect of the disclosure relates to a biological information detecting device including a sensor detecting biological information of a subject; and a case housing the sensor, in which the case includes a top case, and a bottom case, the bottom case includes a light transmitting section including a detection window through which light incident on the sensor is transmitted, and a light shielding section arranged in the vicinity of the light transmitting section, and the light transmitting section extends from the detection window to a sealing portion which is arranged in a connection portion between the top case and the bottom case.

In the aspect of the disclosure, the light transmitting section extends to the sealing portion in the biological information detecting device including the light transmitting section and the light shielding section for receiving and shielding the light with respect to the sensor. Accordingly, it is possible to realize waterproofness between the light transmitting section and the light shielding section by using the sealing portion disposed between the top case and the bottom case.

In the biological information detecting device according to the aspect of the disclosure, the light transmitting section may be formed of a resin material, and the light shielding section may be formed of a glass-containing resin material in which glass is contained in a resin material.

With this configuration, the strength of the light shielding section configuring the case (in particular, the bottom case) is able to increase, and thus it is possible to suppress inflow of moisture or the like due to a modification or the like or damage in internal parts. Further, at the time of increase the strength, it is not necessary to increase the thickness of the member, and thus it is possible to reduce the thickness and the weight of the biological information detecting device.

### BRIEF DESCRIPTION OF THE DRAWINGS

The disclosure will be described with reference to the accompanying drawings, wherein like numbers reference like elements.

FIG. 1 is a sectional view of a biological information detecting device according to an embodiment.

FIGS. 2A and 2B are perspective views of the biological information detecting device of the embodiment.

FIGS. 3A and 3B are plan views of the biological information detecting device according to the embodiment.

FIG. 4 is an explanatory diagram for illustrating that strength is improved by containing glass.

FIG. 5A is a plan view of a packing, and FIG. 5B is a mounting example of the packing.

FIG. 6 is a diagram illustrating an inflow route of moisture.

FIG. 7 is a sectional view of a biological information detecting device of a comparative example.

FIG. 8 is a sectional view of a vicinity of a sensor of a biological information detecting device of another comparative example.

FIG. 9 is a plan view illustrating a portion in which a light shielding section is disposed.

FIGS. 10A and 10B are a sectional view and a plan view illustrating a specific configuration example of the vicinity of the sensor, respectively.

FIG. 11 is a diagram illustrating that signal sensitivity and noise sensitivity are changed by a pressing force.

FIGS. 12A and 12B are diagrams illustrating a height of a convex portion.

FIGS. 13A and 13B are diagrams illustrating the height of the convex portion.

FIG. 14 is an explanatory diagram of a change in the pressing force with respect to weight at the time of disposing a pressing force suppressor.

### DESCRIPTION OF EXEMPLARY EMBODIMENTS

Hereinafter, an exemplary embodiment will be described. Furthermore, this embodiment described below does not unduly limit the contents of the disclosure described in the appended Claims. In addition, it is not limited to that all configurations described in this embodiment are essential configuration requirements of the disclosure.

### 1. Method of this Embodiment

First, a method of this embodiment will be described. As described above, in a wearable biological information detecting device which is mounted on a wrist or the like of a user, a method of acquiring biological information by using a photoelectric sensor is known. As a biological sensor which is the photoelectric sensor, for example, a pulse wave sensor is considered, and it is possible to acquire pulse wave information such as a pulse rate by using the pulse wave sensor.

Hereinafter, a wristwatch type device which is mounted on a wrist will be described as an example, and a biological information detecting device of this embodiment may be mounted on the other portion of the user such as a neck or an ankle. In addition, the biological sensor of this embodiment (the photoelectric sensor) is not limited to the pulse wave sensor, and a photoelectric sensor acquiring biological information other than the pulse wave information may be used. In addition, the biological information detecting device of this embodiment may include a biological sensor other than the photoelectric sensor.

In the biological information detecting device including a photoelectric sensor, it is necessary that necessary light is received and unnecessary light is shielded. According to an example of the pulse wave sensor, reflected light reflected on a subject (in particular, a portion including a blood vessel which is a measurement target) includes a pulse wave component, and thus is received, but the other light becomes a noise component, and thus is shielded. Here, as “the other light”, direct light which is emitted from a light emitting unit and is directly incident on a light receiver, reflected light which is reflected on a portion other than the subject, or environmental light such as sunlight or illumination light is considered.

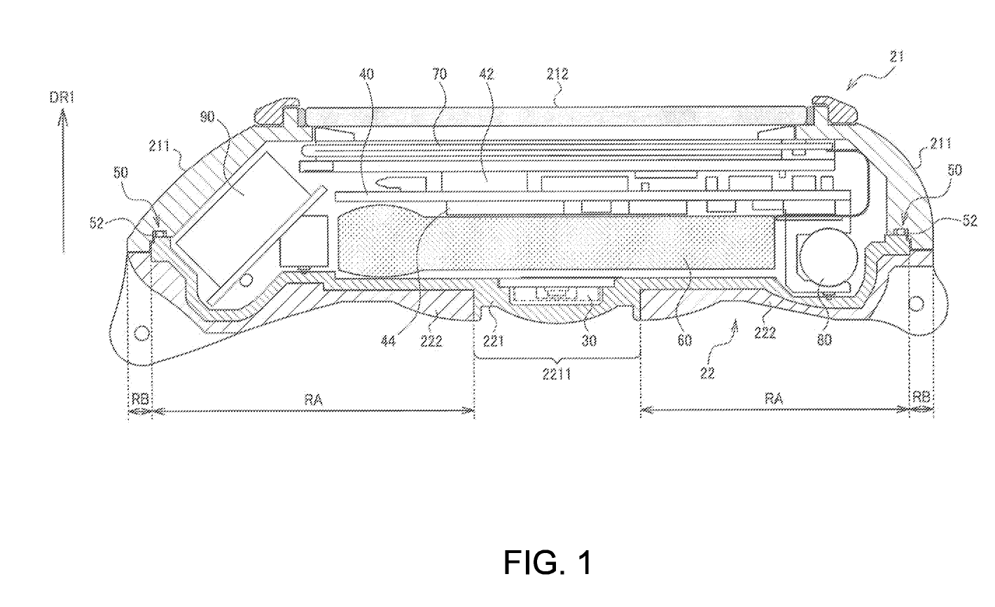

In order to suitably control such light transmitting and light shielding, the biological information detecting device may include a light transmitting section and a light shielding section. As an example, as described below with reference to FIG. 1 or the like, the arrangement or the like of the light transmitting section and the light shielding section in a portion (in a narrow sense, a bottom case) which is disposed on the subject side of the biological information detecting device may be considered.

However, when a use case of the biological information detecting device according to this embodiment is assumed, it is possible to realize a suitable device by satisfying various requirements other than a position relationship between the light transmitting section and the light shielding section.

First, the biological information detecting device has high waterproof properties. In the biological information detecting device (in an example of FIG. 1, a space between a top case 21 and a bottom case 22), a circuit board 40 or a battery (a secondary battery 60), a vibrator 80 (a vibration motor), and the like are included, and thus when the waterproof properties are low, breakdown may occur. In particular, it is also considered that a wearable device such as wristwatch type device is mounted at the time of a movement, and is used for providing information such as movement intensity. In this case, the skin surface of the user may be wet with perspiration, and thus the risk of the inflow of moisture (for example, liquid such as water, or gas such as water vapor) to the device may be suppressed.

Second, it is necessary that the strength of the case (the top case and the bottom case) is high. As described above, various components are arranged in the device, and various forces are applied to the wearable biological information detecting device along with the motion of the user. For example, when the user goes jogging or the like, a pressing force or a twisting force is applied to the device due to a motion such as a vibration of arms. In this case, when a corresponding force is applied to internal parts such as a circuit board, a breakdown occurs in the component.

Third, it is necessary that the mounting feeling of the user becomes excellent. It is necessary that the wearable type biological information detecting device is mounted on the user at the time of being used. When the wearable type biological information detecting device is used at the time of the movement as described above, it is necessary that the device is continuously mounted for a period during which data is desired to be acquired (for example, a period from the start of the movement to the end of the movement). Alternatively, according to an example in which a health degree of the user is determined, it is necessary that the biological information is continuously acquired for a long period of time (for example, a span such as 12 hours, 24 hours, and a few days), and thus the device is mounted during the period. For this reason, it is not preferable that the movement or the daily life of the user is inhibited by mounting the biological information detecting device, and thus excellent mounting feeling becomes an important element. Specifically, the size (the thickness) and the weight of the biological information detecting device may be reduced.

That is, it is preferable that the biological information detecting device is able to receive necessary light and to shield unnecessary light, has high waterproof properties and strength, and is reduced in size and weight. However, in a method of the related art such as JP-A-2012-90975, the structure of the biological information detecting device satisfying these demands is not disclosed.

Therefore, the present inventors have proposed the arrangement and the use material of the light transmitting section and the light shielding section in the structure of a biological information detecting device 1 satisfying the various demands described above, in a narrow sense, in the bottom case. Specifically, the biological information detecting device 1 according to this embodiment, as illustrated in FIG. 1, includes a sensor 30 detecting the biological information of the subject, and a case 20 (to be disposed) housing the sensor 30, and the case 20 includes a light transmitting section 221 including a detection window 2211 through which light incident on the sensor 30 is transmitted, and a light shielding section 222 disposed in the vicinity of the light transmitting section 221. Further, the light transmitting section 221 and the light shielding section 222 are integrally formed, the light transmitting section 221 is formed of a resin material, and the light shielding section 222 is formed of a glass-containing resin material in which glass is contained in a resin material.

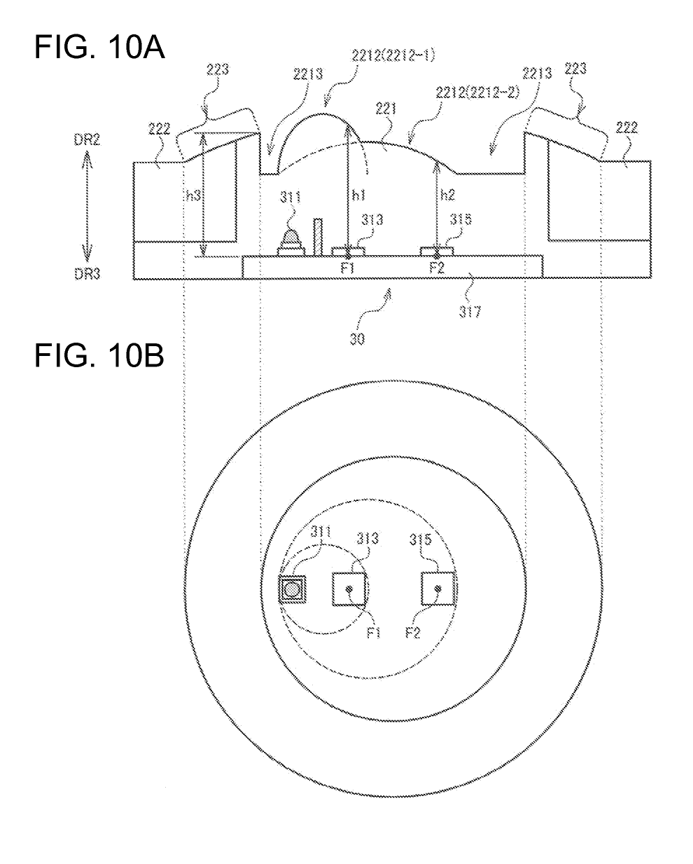

Furthermore, in FIG. 1, the case 20 is configured of the top case 21 and the bottom case 22, and has a structure in which the bottom case 22 includes the light transmitting section 221 and the light shielding section 222, but the configuration is not limited. For example, the case 20 may be formed of an integral member, and various modifications, such as configuring the top plate which is a transparent plate-like member and the case 20 which is formed of a resin member and is combined with the top plate are able to be performed. Hereinafter, the case 20 will be described as having the structure of FIG. 1, and the method of this embodiment is able to be also applied to a case in which the case 20 having another structure including the light transmitting section 221 and the light shielding section 222 is used. In addition, in FIG. 1, the configuration of the peripheral portion of the sensor 30, the detection window 2211, and the detection window 2211 is simplified. A specific structure example will be described with reference to FIG. 10A or the like.

In such a case, light suitable for the photoelectric sensor is able to be used in measurement by the light transmitting section 221 and the light shielding section 222. The light shielding section 222 is formed of a member which does not transmit the light (shields the light), and for example, may shield light from a portion other than the detection window 2211 from being incident on the sensor 30. At this time, when the light transmitting section 221 and the light shielding section 222 are integrally formed, the case 20 (in particular, the bottom case 22) is easily prepared.

With respect to the first demand and the second demand, the case 20 may be rarely modified. When the case 20 is easily modified, an inflow route of liquid or water vapor may be generated due to the modification, or a pressure from the outside may be easily imparted to the internal parts. Here, as the inflow route, a gap between the light transmitting section 221 and the light shielding section 222 is considered. For example, even when the light transmitting section 221 and the light shielding section 222 are formed by two-color molding using a common resin base, the surface is only slightly melted and bonded, and thus water vapor or the like may penetrate a small gap therebetween. However, when the case 20 is rarely modified, it is possible to suppress such a problem. In general, the case 20 is able to be rarely modified by increasing the thickness of the member, but according to this, it is difficult to reduce the size and the weight of the biological information detecting device, and thus it is not possible to respond to the third demand.

From this point, in this embodiment, the light shielding section 222 is formed of the glass-containing resin material, and thus the light shielding section 222 is able to be rarely modified. For this reason, it is not necessary to increase the thickness of the light shielding section 222, and it is possible to reduce the thickness and the weight of the biological information detecting device 1 itself while increasing the waterproof properties and the intensity. That is, the light shielding section 222 is formed of the glass-containing resin material, and thus it is possible to efficiently solve the various problems described above.

Further, by reducing the thickness of the biological information detecting device 1, it is possible to expect an effect of increasing the detection accuracy of the biological information (of suppressing a decrease in the accuracy). This is because, when the biological information detecting device 1 is formed to be thick, the device is in contact with sleeves at the time of wearing long-sleeved clothes, and thus the device vibrates according to the motion of the sleeve. The biological sensor such as the pulse wave sensor is desired to be used by being closely attached to the clothes, but when the device vibrates, the device may become momentarily detached from the user, and thus the measurement accuracy may decrease. From this point, when the thickness of the biological information detecting device 1 is reduced, it is possible to suppress the momentary detachment of the device due to the sleeve or the like, and it is possible to increase the detection accuracy.

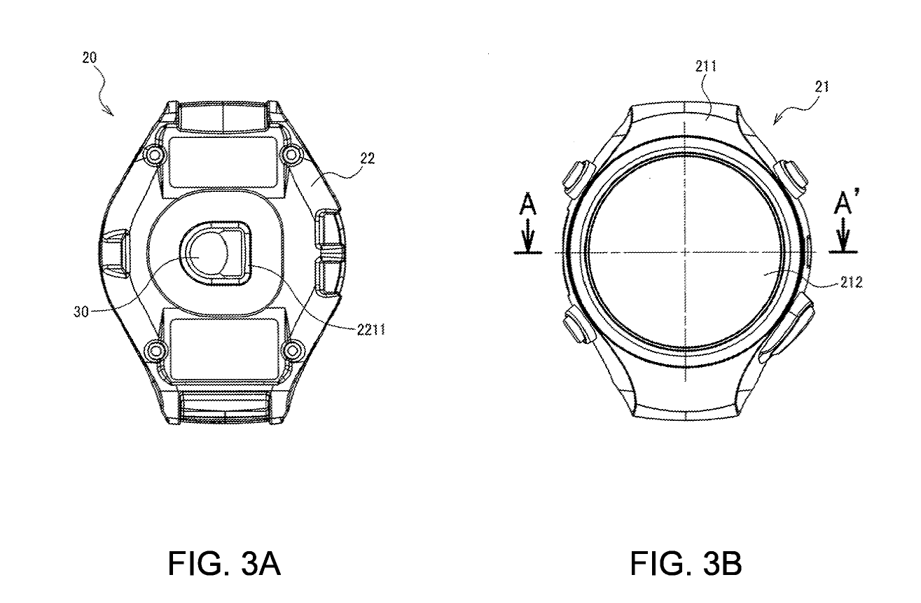

According to such a reduction in the size (the thickness) and the weight, specifically, the weight of the biological information detecting device 1 according to this embodiment is able to be suppressed to be 60 g, an outer case (the case 20) is able to be configured to have a planar size of less than or equal to 6 cm, and a case is able to be configured to have a thickness of less than or equal to 15 mm. Here, as in FIG. 3A described below, the planar size of the case 20 indicates a size in a plan view in a direction observed from the subject (the wrist of the user) side in a state where the biological information detecting device 1 is mounted on the user, and the case thickness indicates a size in a direction orthogonal to the case (for example, a DR1 direction in FIG. 1). Specifically, the maximum value of the length of the case 20 in a plan view is able to be less than or equal to 6 cm, and the maximum value of the thickness in the DR1 direction is able to be less than or equal to 15 mm.

In addition, for example, it is assumed that measurement of a movement situation and measurement of a health degree shown in the third demand described above are included as the use case of the biological information detecting device 1 according to this embodiment. Accordingly, the present applicant has realized the biological information detecting device 1 which is able to be continuously used for a long period of time by considering the configuration of the sensor 30, the circuit board 40, or the other component, and a control method. As the secondary battery 60 described below, a specific example will be described in which a battery of 150 mAh is used.

A driving time is also changed according to an operation situation. For example, when positioning per second of a GPS (a GPS antenna 90 described below), and measurement of the pulse wave information of a pulse wave sensor 31 (the sensor 30) are concurrently performed, the biological information detecting device 1 is able to be driven for 20 hours. In addition, when the pulse wave sensor 31 is set to be OFF, and the positioning per second of the GPS is performed, the biological information detecting device 1 is able to be driven for 24 hours. In addition, when the GPS is set to be OFF, and the measurement of the pulse wave sensor 31 is performed, the biological information detecting device 1 is able to be driven for 60 hours.

Hereinafter, a specific configuration example of the biological information detecting device 1 of this embodiment will be described with reference to FIG. 1 to FIG. 5B or the like. After that, as a specific example of the sensor 30 according to this embodiment, a pulse wave sensor including two light receivers will be described, and finally, a modification example of this embodiment will be described.

### 2. Configuration of Biological Information Detecting Device

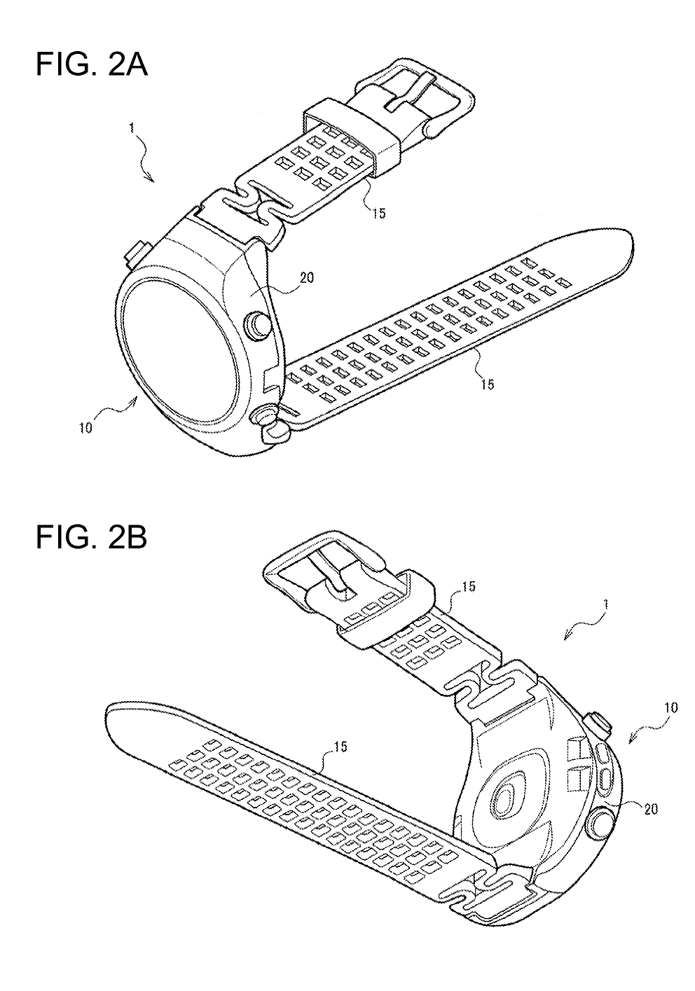

In FIGS. 2A and 2B, a perspective view of the biological information detecting device 1 according to this embodiment is illustrated. FIG. 2A is a perspective view seen from the top case 21 side, and FIG. 2B is a perspective view seen from the bottom case 22 side. The biological information detecting device 1 according to this embodiment is mounted on a predetermined portion of the user (for example, the wrist), and detects the biological information such as the pulse wave information. The biological information detecting device 1 includes a device main body 10 which is closely attached to the user and detects the biological information, and a band portion 15 for mounting the device main body 10 on the user by being attached to the device main body 10.

The device main body 10 includes the top case 21 and the bottom case 22. FIGS. 3A and 3B are diagrams illustrating a portion of the device main body 10 in the biological information detecting device 1. FIG. 3A is a plan view in a direction from the bottom case 22 to the top case 21, that is, a direction observed from the subject (the wrist of the user) side in a state where the biological information detecting device 1 is used by being mounted on the user. In addition, FIG. 3B is a plan view on a side opposite to that of FIG. 3A, that is, a direction from the top case 21 to the bottom case 22. That is, FIG. 3A is a plan view mainly illustrating the structure of the bottom case 22, and FIG. 3B is a plan view mainly illustrating the structure of the top case 21.

As illustrated in FIG. 3A, the detection window 2211 is disposed in the bottom case 22, and the sensor 30 is disposed in a position corresponding to the detection window 2211. In the detection window 2211, light emitted from the light emitting unit 311 in the photoelectric sensor (the pulse wave sensor 31) which transmits light and is disposed in the sensor 30 is transmitted through the detection window 2211 and is emitted to the subject. In addition, reflected light on the subject is also transmitted through the detection window 2211, and is received in the light receiver (for example, a first light receiver 313 and a second light receiver 315 which will be described below with reference to FIG. 10A or the like) in the pulse wave sensor 31. That is, by disposing the detection window 2211, the biological information is able to be detected by the photoelectric sensor. Specifically, the detection window 2211 may be realized by the light transmitting section 221 (the light transmitting section 221 may include the detection window 2211). A specific structure of the light transmitting section 221 will be described below.

As illustrated in FIG. 3B, the top case 21 may include a body portion 211 and a glass plate 212. In this case, the body portion 211 and the glass plate 212 may be used as an outer wall protecting an internal structure, and a display of a display unit such as a liquid crystal display (an LCD 70) which is disposed immediately under the glass plate 212 may be browsed by the user through the glass plate 212. That is, in the biological information detecting device 1 of this embodiment, various information items such as information indicating detected biological information or a movement state or time information may be displayed by using the LCD 70, and the display may be provided to the user from the top case 21 side. Furthermore, here, an example is described in which a top plate portion of the biological information detecting device 1 is realized by the glass plate 212, and when the LCD 70 is a transparent member which is able to be browsed, and is a member having strength to the extent of protecting a configuration included in the case 20 such as the LCD 70, the top plate portion is able to be configured of a material other than the glass, such as transparent plastic.

Next, an example of the specific sectional structure of the device main body 10 in the biological information detecting device 1 will be described with reference to FIG. 1. FIG. 1 is a sectional view cut along line A-A′ of FIG. 3B, an upper side of the paper in FIG. 1 is the top case 21 side, and a lower side of the paper is the bottom case 22 side.

As illustrated in FIG. 1, the device main body 10 includes the sensor 30, the circuit board 40, a panel frame 42, a circuit case 44, the secondary battery 60, the LCD 70, the vibrator (the vibration motor) 80, and the GPS antenna 90 in addition to the top case 21 and the bottom case 22. However, the configuration of the biological information detecting device 1 is not limited to that of FIG. 1, and other configurations are able to be added, or a part of the configuration is able to be omitted. For example, the GPS antenna 90 may be omitted from the configuration of FIG. 1.

The sensor 30 includes the photoelectric sensor. According to this, the biological information detecting device 1 is formed by including the photoelectric sensor in the sensor 30, and thus, according to these properties, for example, a pulse wave is measured as the biological information, and on the basis of this information, a state relevant to a pulse rate or stiffness of a blood vessel, and a movement, a psychological state, or the like is able to be derived.

The photoelectric sensor collects the light which is emitted towards the wrist of the user from the light emitting unit 311 such as a Light Emitting Diode (LED) and is reflected on the blood vessel of the wrist by a light collecting mirror, and receives the light by the light receiver such as a photodiode. At this time, the photoelectric sensor measures a pulse of the user by using a phenomenon in which a reflection ratio of the light is different between expansion and contraction of the blood vessel. From this, it is preferable that the sensor 30 is pressed with respect to the wrist such that light which is a measurement noise is not received by the light receiving device of the photoelectric sensor, and it is more preferable that the sensor 30 is closely attached to the wrist.

Furthermore, a specific configuration example of the sensor 30 will be described below with reference to FIG. 10A or the like. In addition, in consideration of applying a suitable pressing force, a convex portion 2212 for applying a pressing force to the light transmitting section 221 may be disposed, and the convex portion 2212 will be described below with reference to FIG. 10A or the like.

In the circuit board 40, the panel frame 42 guiding a display panel such as the LCD 70 is arranged in one surface, and a circuit case 44 guiding the secondary battery 60 or the like is arranged in the other surface.

Furthermore, an epoxy resin-based substrate containing a glass fiber, and the like are used as the circuit board 40, and a wiring pattern formed of a copper foil is formed on both surfaces of the circuit board 40. In addition, a resin such as a polyacetal or a polycarbonate is used as the panel frame 42 and the circuit case 44.

In the circuit board 40, a device configuring a circuit which drives the photoelectric sensor and measures the pulse, a circuit which drives the LCD 70, a circuit which controls each of the circuits, and the like are mounted. The circuit board 40 includes an electrode for being connected to the LCD 70 on one surface, and is electrically conductive to the electrode of the LCD 70 through a connector (not illustrated).

In the LCD 70, pulse measurement data such as a pulse rate, time information such as a current time, and the like are displayed according to each mode.

In the circuit case 44, a button type secondary battery 60 (a lithium secondary battery) which is able to be charged is stored. Terminals of both electrodes of the secondary battery 60 are connected to the circuit board 40, and supply a power source to a circuit controlling the power source. The power source is supplied to each of the circuits by being converted into a predetermined voltage in this circuit, and operates the circuit which drives the photoelectric sensor and detects the pulse, the circuit which drives the LCD 70, the circuit which controls each of the circuits, and the like. The secondary battery 60 is charged through a pair of charge terminals which is electrically conductive to the circuit board 40 by a conductive member such as a coil spring. Furthermore, here, an example is described in which the secondary battery 60 is used as the battery, and a primary battery in which the charge is not necessary may be used as the battery.

As described above, the biological information detecting device 1 according to this embodiment includes the secondary battery 60 housed in the case 20, and the circuit board 40 electrically connected to the sensor 30, as illustrated in FIG. 1. Then, the secondary battery 60 is arranged between the circuit board 40 and the sensor 30. Here, the circuit board 40 may be a substrate on which a processing device of the biological information detecting device 1 is mounted. Here, the secondary battery 60 and the circuit board 40 may be disposed in the center portion of the biological information detecting device 1 in a plan view (corresponding to FIG. 3A) seen from a contact surface side with respect to the subject.

In addition, in the biological information detecting device 1, the vibrator 80 (the vibration motor) may be disposed between the secondary battery 60 and the case 20 in a plan view the case 20 (in a narrow sense, the bottom case 22) is seen from a direction perpendicular to the contact surface with respect to the subject. Furthermore, here, the direction perpendicular to the contact surface may be direction from the bottom case 22 towards the top case 21 (DR1 in FIG. 1), or an opposite direction thereof. The vibrator 80, for example, may perform any notification to the user, or is able to be used as a user interface different from the LCD 70. In the example of FIG. 1, the vibrator 80 is disposed on a right end side from the secondary battery 60.

Next, the detail of the sectional structure of the light transmitting section 221 and the light shielding section 222 will be described. As known from FIG. 1, the light shielding section 222 is disposed to cover the light transmitting section 221 from the subject side in the portion other than the detection window 2211.

In the detection window 2211, the light transmitting section 221 is not covered with the light shielding section 222, in other words, the detection window 2211 is realized by the light transmitting section 221. For this reason, as described above, the photoelectric sensor disposed in the sensor 30 is able to emit light with respect to the subject from the light emitting unit 311, or is able to receive the light reflected on the subject in the light receiver (the first light receiver 313 and the second light receiver 315), and thus it is possible to detect the biological information such as the pulse wave information.

On the other hand, in the portion other than the detection window 2211, the light transmitting section 221 is covered with the light shielding section 222 from the subject side (the lower side of the paper in FIG. 1). According to this, it is possible to limit the light incident on the sensor 30. For this reason, light which is desired to receive, that is, the reflected light which is emitted from the light emitting unit 311 and is reflected on the subject is able to be received, and the light which is the noise source, for example, environmental light such as solar light or illumination light is able to be prevented from being received, and thus it is possible to improve the detection accuracy of the biological information.

In addition, the structure in which the light transmitting section 221 is covered with the light shielding section 222 is able to be grasped from other viewpoints. Specifically, in the biological information detecting device 1 of this embodiment, in a state where the biological information detecting device 1 is mounted on the user (the subject), the light transmitting section 221 is disposed on a first direction DR1 side of the light shielding section 222 in the portion other than the detection window 2211 when a direction from the subject towards the case 20 (in a narrow sense, the direction from the bottom case 22 to the top case 21) is the first direction DR1.

Insofar as the light transmitting section 221 transmits the light, in a portion where the light transmitting section 221 is disposed, a possibility of inflow of the light through the portion has to be considered. Here, the light transmitting section 221 is disposed in the bottom case 22, and thus an incident direction of the light to be considered is the direction from the subject towards the bottom case 22, that is, the first direction DR1. At this time, when the light transmitting section 221 is disposed on the DR1 side of the light shielding section 222, it is considered that the light to the light transmitting section 221 other than the detection window 2211 is affected by the light shielding of the light shielding section 222, and thus it is possible to prevent the light which is the noise source from being incident on the sensor 30.

Furthermore, as known from the example of FIG. 1, disposing the light transmitting section 221 on the DR1 side of the light shielding section 222 does not indicate that the light transmitting section 221 is disposed on the DR1 side from an entire region of the light shielding section 222. For example, as with a region indicated by RB in FIG. 1, there may be a region in which the light transmitting section 221 is not arranged on the DR1 side of the light shielding section 222. That is, disposing the light transmitting section 221 on the DR1 side of the light shielding section 222 may indicate that the light shielding section 222 is disposed on a direction side opposite to DR1 excluding the portion of the detection window 2211 when the light transmitting section 221 is disposed. Specifically, in a region indicated by RA in FIG. 1, that is, in a region in which the light transmitting section 221 other than the detection window 2211 is disposed, the light transmitting section 221 is disposed on the DR1 side from the light shielding section 222.

In other words, in the biological information detecting device 1 according to this embodiment, the light shielding section 222 is disposed to be superimposed on the light transmitting section 221 from the subject side of the light transmitting section 221 in the portion other than the detection window 2211. That is, in a portion in which the light shielding section 222 is superimposed on the light transmitting section 221 from the subject side, the light from the outside to the inside of the case 20 is shielded by the light shielding section 222, and the light to the inside of the case 20 (in a narrow sense, the sensor 30) is incident on a portion in which the light shielding section 222 is not superimposed on the light transmitting section 221. For this reason, as described above, the light is able to be transmitted through the portion of the detection window 2211 and is able to be shielded by the other portion.

Here, the light transmitting section 221 is formed of the resin material, and the light shielding section 222 is formed of the glass-containing resin material containing glass (in a narrow sense, a glass fiber). Specifically, the light transmitting section 221 contains any one of a polycarbonate, an ABS resin, and an acrylic resin, and the light shielding section 222 contains any one of a polycarbonate containing glass, an ABS resin containing glass, and an acrylic resin containing glass.

That is, the light shielding section 222 according to this embodiment may be formed of Fiber Reinforced Plastics (FRP), and in particular among them, may be Glass Fiber Reinforced Plastics (GFRP) using a glass fiber as a fiber which is used for reinforcement. In GFRP, a thermoplastic resin may be used as a resin used along with the glass fiber, and in this embodiment, a polycarbonate or an ABS resin is able to be used as the thermoplastic resin. In addition, it is known that an acrylic resin includes a thermoplastic acrylic resin and a thermosetting acrylic resin, and in this embodiment, any one thereof is able to be used. GFRP is cheap among FRP and is general, and thus by adopting GFRP, it is possible to easily realize the light shielding section 222 according to this embodiment. Furthermore, as the resin material of GFRP, various resin materials such as a polyester resin, a vinyl ester resin, an epoxy resin, and a phenol resin are able to be used, and thus the light shielding section 222 according to this embodiment is able to widely use these materials. For example, a resin material which is a target containing glass is not limited to each independently using a polycarbonate, an ABS resin, and an acrylic resin, and a modification of using an alloy material in which these resins are crossed is able to be performed.

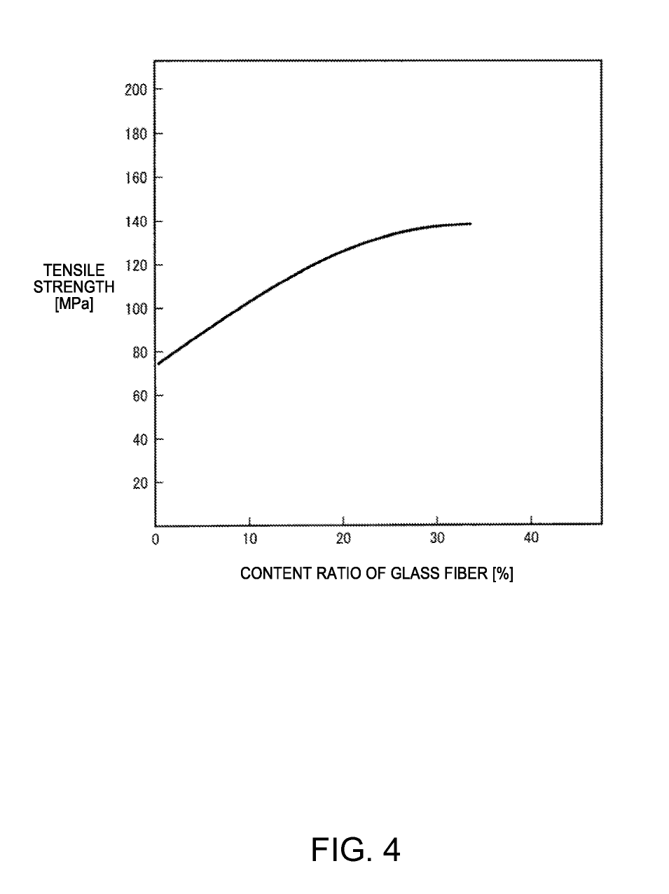

In FIG. 4, a graph illustrating a relationship between a content ratio of the glass fiber and tensile strength of GFRP is illustrated. FIG. 4 is an example of a case where, in GFRP, the polycarbonate is used, in particular as the resin material. As known from FIG. 4, it is possible to increase the strength by containing glass, and as described above, a waterproof effect due to the suppression or an effect of suppressing impact with respect to the internal parts is able to be expected by suppressing a modification.

In addition, in the biological information detecting device 1 of this embodiment, at least the light shielding section 222 may be formed of a glass-containing resin material, and thus the light transmitting section 221 may be formed of a resin material which does not contain glass. However, the configuration is not limited thereto, and the light transmitting section 221 may be formed of a glass-containing resin material.

As described above, it is necessary that the light transmitting section 221 transmits the light, and in general, it is considered that transparency (light transmittance) is decreased by containing the glass fiber. However, recently, a glass-containing resin material which is able to increase the strength while maintaining high transparency is developed. The light transmitting section 221 may be formed by using such a glass-containing resin material having high transparency, and in this case, it is possible to increase the strength of the light transmitting section 221 without considerably decreasing the light transmittance. Furthermore, even when the light transmittance decreases, and thus the detection accuracy of the sensor slightly decreases, these decreases may not be particularly problematic depending on the usage of the biological information detecting device 1, the biological information which is a detection target, or the like. In this case, without using the glass-containing resin material having high transparency, the light transmitting section 221 may be formed of a general glass-containing resin material (having low transparency compared to the resin material which does not contain glass or the glass-containing resin material having high transparency). In this example, the light transmittance slightly decreases, but the light transmitting section 221 having high strength is able to be easily formed.

Furthermore, various embodiments are considered as the resin material forming the light transmitting section 221, and as described above, the light transmitting section 221 may be formed of a polycarbonate, an ABS resin, and an acrylic resin, or may be formed of an alloy material in which these resins are crossed. A transparent resin material is used for forming the light transmitting section 221, and as the transparent resin material, for example, an alloy material in which a polycarbonate and an acrylic resin are crossed is widely known.

In addition, it is described that the light transmitting section 221 and the light shielding section 222 are integrally formed, and here, the light transmitting section 221 and the light shielding section 222 may be integrally formed by two-color molding or insert molding. In other words, the light transmitting section 221 and the light shielding section 222 may be integrally formed by two-color molding or insert molding.

Furthermore, the two-color molding and the insert molding have a common point that different materials (materials) are integrally molded by being combined. A different point is that in the two-color molding, a portion which is a primary side is molded, and a portion which is a secondary side is integrally molded with the primary side in the same metal mold, but in the insert molding, the portion which is the primary side is taken out from the mold after the molding, and the taken-out component is set in the mold of the secondary side and is integrally molded with the portion which is the secondary side. The bottom case 22 according to this embodiment may use any molding method, and when mass production is assumed, it is not necessary to detach the primary side component from the metal mold, and thus the two-color molding is advantageous.

In addition, integrally forming the light transmitting section 221 and the light shielding section 222 is not limited to integrating two members at the time of forming each unit. That is, the light transmitting section 221 and the light shielding section 222 according to this embodiment are not limited to being formed by the two-color molding or the insert molding, and may be integrally formed by separately molding the light transmitting section 221 and the light shielding section 222, and then by adhering or welding the light transmitting section 221 to the light shielding section 222.

In addition, as illustrated in FIG. 1, the light transmitting section 221 may be formed to extend from the detection window 2211 to a sealing portion 50 disposed on a connection portion between the top case 21 and the bottom case 22. Here, in the sealing portion 50, a packing 52 sealing the inside of the case 20 from the outside may be disposed.

As described above, in the biological information detecting device 1 assumed in this embodiment, the case 20 is realized by combining the top case 21 with the bottom case 22, and various components such as the circuit board 40 are housed in the case 20. That is, the connection portion (a gap) between the top case 21 and the bottom case 22 is the inflow route of moisture or the like, and thus in order to increase the waterproof properties of the biological information detecting device 1, the sealing portion 50 sealing the gap is necessarily disposed.

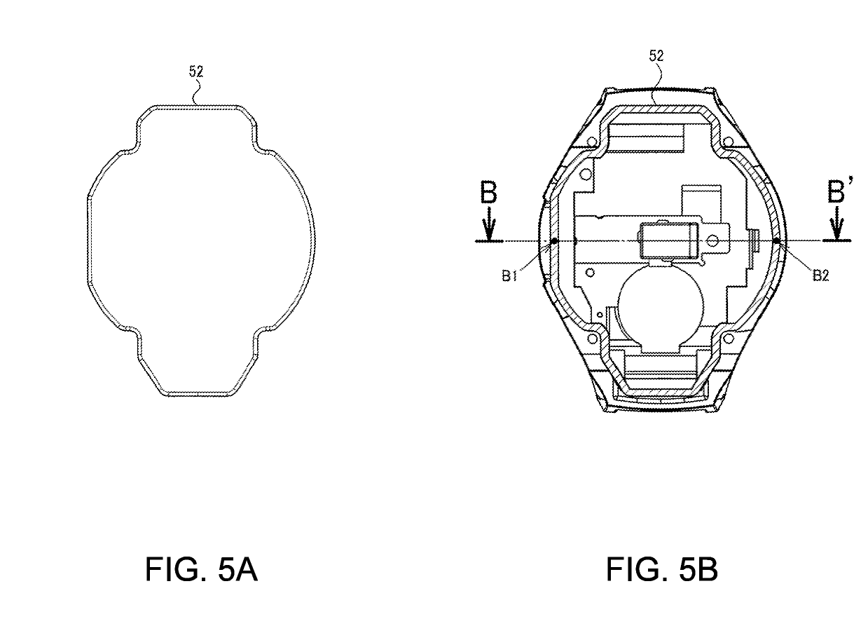

In FIGS. 5A and 5B, a plan view of the packing 52 is illustrated. As with FIG. 3A, FIGS. 5A and 5B are plan views in which the device main body 10 is seen from the bottom case 22 side. However, in order to draw the packing 52, FIG. 5B is a diagram further illustrating the structure of the inside of the device main body 10 compared to FIG. 3A.

Each of the top case 21 and the bottom case 22 is connected to a circumferential edge portion in a plan view of FIG. 3A, FIG. 3B, and the like, and thus the packing 52 is disposed to cover the circumferential edge portion. Specifically, the packing 52 may be realized by a closed curve as in FIG. 5A. An example in which the packing 52 illustrated in FIG. 5A is actually mounted on the device is FIG. 5B. In this case, FIG. 1 is a sectional view cut along line B-B′ of FIG. 5B, and thus portions indicated by B1 and B2 in the packing 52 of FIG. 5B are observed as the packing 52 of FIG. 1.

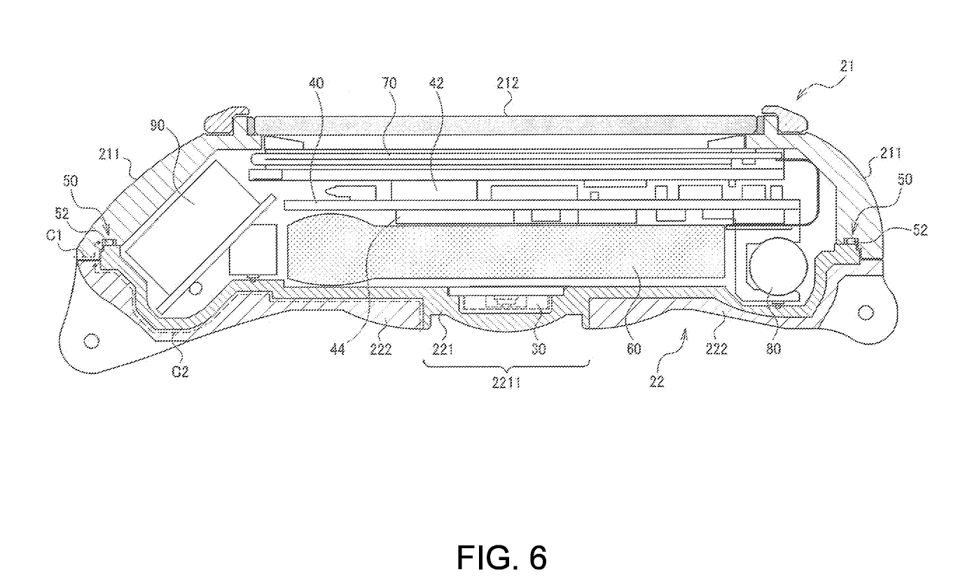

As described above, this packing 52 is disposed on the connection portion between the top case 21 and the bottom case 22, and seals the inside of the case 20 from the outside. Specifically, the packing 52 suppresses the inflow of the moisture or the like in a route indicated by C1 of FIG. 6. At this time, when the light transmitting section 221 is formed to extend to the position of the packing 52, the packing 52 is able to suppress the inflow of the moisture or the like in a route indicated by C2. That is, as illustrated in FIG. 1, the light transmitting section 221 is formed to extend, and thus the packing 52 which is used in waterproofing of the connection portion between the top case 21 and the bottom case 22 is able to be also used in the waterproofing between the light transmitting section 221 and the light shielding section 222.

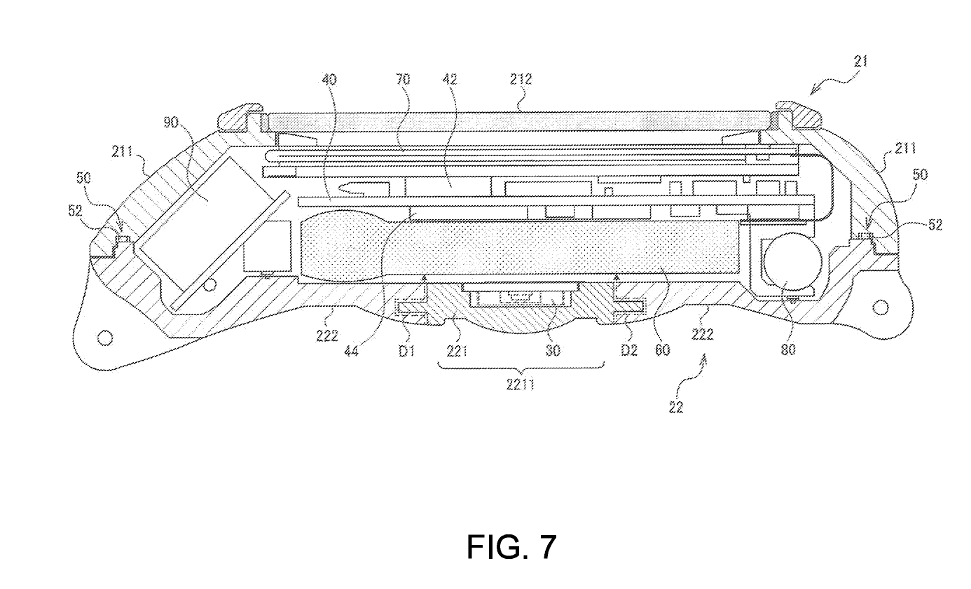

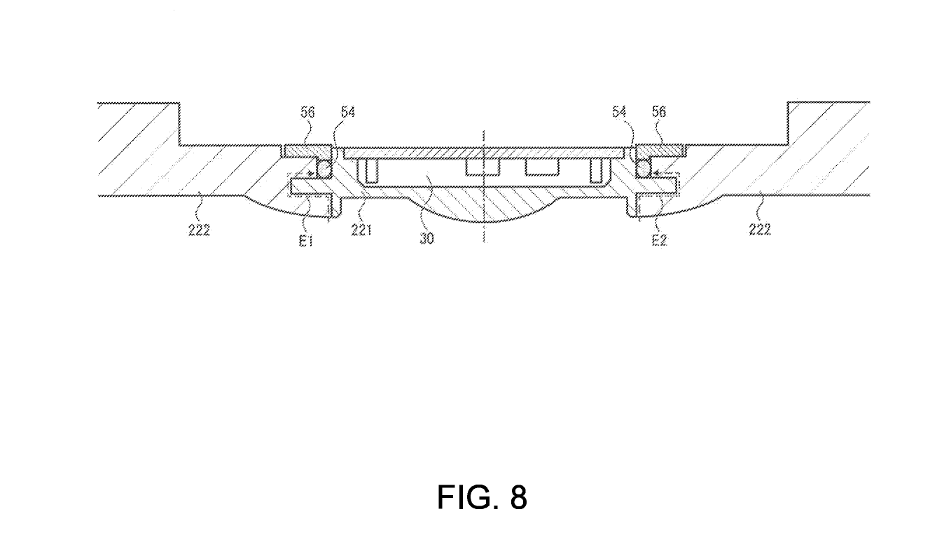

Accordingly, in comparison with the comparative example of this embodiment illustrated in FIG. 7 and FIG. 8, it is possible to increase waterproof properties by a simple structure without disposing a new structure for waterproofness such as the sealing portion. FIG. 7 is an example in which the light transmitting section 221 is not formed to extend, and the moisture or the like may inflow due to a route of D1 or D2 of FIG. 7. Furthermore, FIG. 7 is a sectional view in which the device main body 10 is seen from the direction as that of FIG. 1.

In addition, FIG. 8 is a sectional view in which the vicinity of the bottom case 22 and the sensor 30 of the device main body 10 is seen from the same direction as that of FIG. 1. In FIG. 8, as with FIG. 7, the light transmitting section 221 is not formed to extend, and in order to suppress the inflow of the moisture or the like in a route of E1 or E2 (corresponding to D1 or D2 in FIG. 7), a packing 54 is added to the configuration of the light transmitting section 221. According to such a configuration, the waterproof properties are ensured. However, in order to increase the waterproof properties by the structure of the packing 54 or the like, in general, it is necessary to apply a force to the packing 54 or the like and press the packing 54. Specifically, as illustrated in FIG. 8, it is necessary to dispose a member 56 for pressing the packing 54. That is, in an example of FIG. 8, a new member such as the member 56 for increasing the waterproof properties is added, and thus the structure of the device main body 10 becomes complicated, and thus it is difficult to reduce the thickness of the device main body 10 due to an influence of the added member. As described above, the reduction in the thickness is an important element relevant to the mounting feeling of the user, and thus adding the member as in FIG. 8 is not preferable.

From this point, according to the method of this embodiment illustrated in FIG. 1, an existing packing 52 is able to be used, and thus it is possible to efficiently increase the waterproof properties by a simple structure, and it is not difficult to realize the reduction in the thickness.

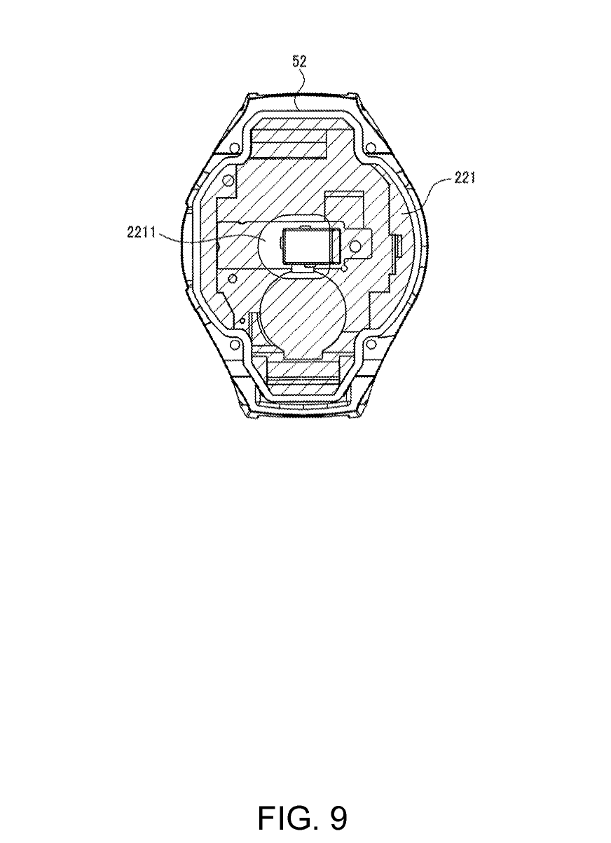

Furthermore, as forming the light transmitting section 221 to extend form, in other words, it is able to be considered that the light transmitting section 221 is disposed in a region between the detection window 2211 and the sealing portion 50 in a plan view in which the bottom case 22 is seen from the direction perpendicular to the contact surface with respect to the subject. Specifically, as with FIG. 3A, FIG. 9 is a plan view in which the device main body 10 is seen from a direction perpendicular to the subject (in particular, the direction from the subject towards the device main body 10), and the light transmitting section 221 is disposed in a portion indicated by hatched lines of FIG. 9. Further, as illustrated in FIG. 1, the detection window 2211 is also realized by the light transmitting section 221, and thus it may be considered that the light transmitting section 221 is disposed in a region on the inside of the bottom case 22 from the sealing portion 50. However, it is not necessary that the entire region on the inside from the sealing portion 50 is covered with the light transmitting section 221, and a modification of not disposing the light transmitting section 221 in a part of the region is able to be performed.

### 3. Configuration Example of Sensor and Peripheral Structure Example of Detection Window

A configuration example of the sensor 30 according to this embodiment will be described. The sensor 30 may include the first light receiver 313 and the second light receiver 315 receiving the light from the subject. Then, in a direction from the biological information detecting device 1 to the subject, when the height in a position or a region of the light transmitting section 221 corresponding to the first light receiver 313 is h1, and the height in a position or a region of the light transmitting section 221 corresponding to the second light receiver 315 is h2, h1>h2 may be satisfied.

This configuration is illustrated in FIGS. 10A and 10B, and FIGS. 10A and 10B are sectional views in which the vicinity of the sensor 30 is seen from the same direction as that of FIG. 1. Furthermore, in FIGS. 10A and 10B, for the sake of simplicity, the configuration of the biological information detecting device 1 according to this embodiment (in particular, the height or the shape of the light transmitting section 221) is schematically illustrated, and the dimension or the ratio in the drawing is different from the actual dimension and ratio.

When the biological information such as the pulse wave information is detected by using the photoelectric sensor, a noise due to a body motion becomes a problem. For this reason, in order to detect the biological information with high accuracy, it is necessary to reduce the body motion noise by using any method.

When the body motion noise is reduced, a component corresponding to a pulse signal in a detection signal of the photoelectric sensor is maintained at the most, and a component corresponding to the body motion noise is reduced (in a narrow sense, eliminated). That is, in processing of reducing the body motion noise, it is necessary to know which signal component corresponds to the body motion noise.

In contrast, a method of reducing the body motion noise by using a motion sensor is known. The motion sensor is a sensor detecting the motion of the user (a person mounting the biological information detecting device 1), and thus a signal corresponding to the body motion, that is, a signal corresponding to the body motion noise is able to be acquired by using the motion sensor. Here, as the motion sensor, for example, an acceleration sensor, a gyro sensor, an atmospheric pressure sensor, and the like are considered.

In this embodiment, the method of reducing the body motion noise by using the motion sensor described above may be used together, and here, a signal including a lot of body motion noises is acquired by using the second light receiver 315 different from the first light receiver 313 detecting the pulse signal. As described above, the body motion noise is included in the detection signal of the photoelectric sensor. By using this point, in the second light receiver 315, intentionally, the sensitivity of the pulse signal is set to be low, and the sensitivity of the body motion noise is set to be high, and thus it is possible to acquire the detection signal mainly including body motion noise.

When the signal corresponding to the body motion noise is detected in the second light receiver 315, a component corresponding to the detection signal of the second light receiver 315 is eliminated (reduced) from the detection signal of the first light receiver 313, and thus it is possible to reduce the body motion noise. At this time, the sensitivity of the pulse signal decreases in the second light receiver 315, and thus is not excessively reduced to a pulse component included in the detection signal of the first light receiver 313.

However, in order to execute such processing, it is necessary that properties of the body motion noise included in the detection signal (for example, frequency properties) are coincident with each other (or sufficiently close to each other) in the first light receiver 313 and the second light receiver 315. That is, a correlation in the detection signals of the two light receivers has to be maintained to be high while maintaining a difference in the detection properties such that the first light receiver 313 mainly detects the pulse signal and the second light receiver 315 mainly detects the body motion noise.

It is known that sensitivity with respect to the pulse signal or the body motion noise is changed according to the pressing force with respect to the subject, and thus in this embodiment, a difference is assumed to be provided between a pressing force corresponding to the first light receiver 313 and a pressing force corresponding to the second light receiver 315.

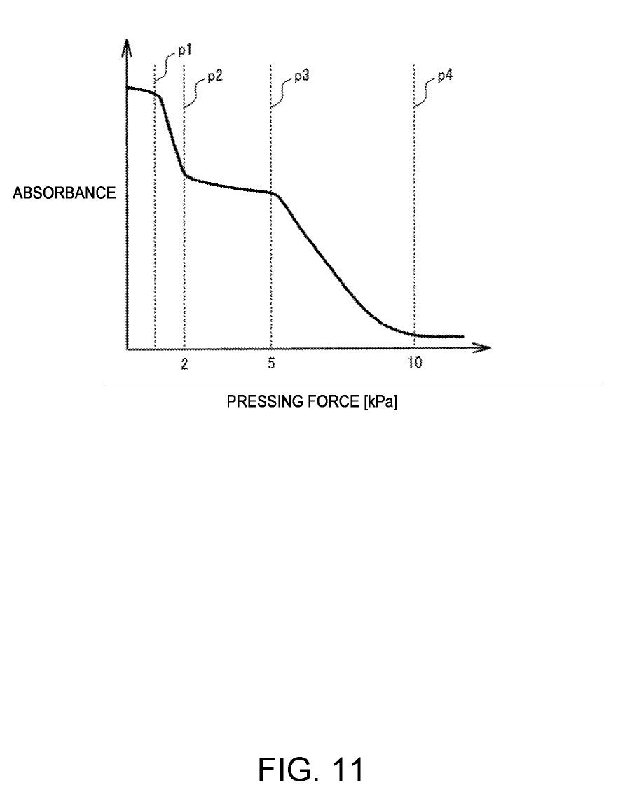

FIG. 11 is a diagram exemplifying a change in absorbance with respect to the pressing force. A horizontal axis indicates the pressing force, and a vertical axis indicates the absorbance of the blood vessel. When the pressing force is changed, the blood vessel which is subjected to the influence of the pressing force is changed. A blood vessel which is most easily subjected to the influence, that is, a blood vessel which is subjected to the influence of the lowest pressing force is a capillary blood vessel. In an example of FIG. 11, when the pressing force exceeds p1, a change amount of the absorbance increases, and this indicates that the capillary blood vessel starts to collapse by the pressing force. When the pressing force exceeds p2, the change in the absorbance becomes smooth, and this indicates that the capillary blood vessel approximately completely collapses (is closed). A blood vessel which is subjected to the influence next to the capillary blood vessel is an artery. Further, when the pressing force increases and exceeds p3, the change amount of the absorbance increases again, and this indicates that the artery starts to collapse by the pressing force. When the pressing force exceeds p4, the change in the absorbance becomes smooth, and this indicates that the artery approximately completely collapses (is closed).

In this embodiment, the second light receiver 315 detects a signal corresponding to the capillary blood vessel, and thus the ratio of the body motion noise increases, and the first light receiver 313 measures a signal corresponding to the artery (a pulse signal), and thus the ratio of the pulse signal increases. For this reason, the pressing force in the second light receiver 315 is designed to be in a range of p1 to p2, and the pressing force in the first light receiver 313 is designed to be in a range of p3 to p4. It is preferable that a difference in the pressing force of the first light receiver 313 and the second light receiver 315, for example, is greater than or equal to 2.0 kPa and less than or equal to 8.0 kPa.

The difference in the pressing force, specifically, may be realized by a difference in the height of the subject and the light transmitting section 221 which is in contact with the subject. As described above, the pressing force increases in the first light receiver 313 mainly detecting the pulse signal, and the pressing force decreases in the second light receiver 315, compared to the first light receiver 313. For this reason, the height h1 of the light transmitting section 221 in the position or the region corresponding to the first light receiver 313 may be higher than the height h2 of the light transmitting section 221 in the position or the region corresponding to the second light receiver 315.

This is because, here, the first light receiver 313 protrudes to the subject side as the height becomes higher, and thus when the biological information detecting device 1 is fixed to the wrist or the like by a predetermined cuff pressure, the pressing force corresponding to the first light receiver 313 having a high height becomes stronger than the pressing force corresponding to the second light receiver 315 having a low height.

Specifically, the light transmitting section 221 includes the convex portion 2212, and a suitable pressing force is applied to the subject by the convex portion 2212. Then, in the biological information detecting device 1 according to this embodiment, a plurality of photoelectric sensors is realized by disposing a plurality of light receivers, and thus a plurality of convex portions 2212 (for example, the number corresponding to the number of photoelectric sensors) may be disposed. In an example of FIG. 10A, a convex portion 2212-1 is disposed in the first photoelectric sensor realized by the light emitting unit 311 and the first light receiver 313, and a convex portion 2212-2 is disposed in the second photoelectric sensor realized by the light emitting unit 311 and the second light receiver 315.

At this time, in a state where the biological information detecting device 1 is mounted, when the direction from the sensor 30 towards the subject (DR2 in FIG. 10A) is a height direction, the height h1 of the light transmitting section 221 in the position or the region corresponding to the first light receiver 313 is higher than the height h2 of the light transmitting section 221 in the position or the region corresponding to the second light receiver 315. This, for example, is able to be realized by setting the height of the convex portion 2212-1 to be higher than the height of the convex portion 2212-2. Furthermore, the definition of the height is able to be variously modified, and for example, as illustrated in FIG. 10A, a distance from a surface of a sensor substrate 317 on which the light emitting unit 311 and the like are disposed may be the height. Alternatively, the thickness of the light transmitting section 221 may be the height.

Alternatively, in a state where the biological information detecting device 1 is mounted, a reference surface may be set to be disposed on a side (a lower side in FIG. 10A) opposite to the subject with respect to the sensor substrate 317 and to be in parallel with the surface of the sensor substrate 317, and a distance from the reference surface may be the height of the light transmitting section 221. The reference surface may be a surface of any member (for example, the circuit board 40), or may be an imaginary surface.

In addition, the definition of the position or the region corresponding to the light receiver is variously considered. For example, the height h1 may be the height of the light transmitting section 221 in the representative position of the first light receiver 313, and the height h2 may be the height of the light transmitting section 221 in the representative position of the second light receiver 315. Here, as the representative position, for example, the center position of each of the light receivers or the like may be used.

In this case, the center position of the first light receiver 313 is F1 in FIG. 10B, and the center position of the second light receiver 315 is F2. Then, as illustrated in FIG. 10A, the height of the light transmitting section 221 in the center portion F1 of the first light receiver 313 may be defined as an intersection point between a straight line extending in the DR2 direction from F1 and the surface of the light transmitting section 221 (a surface which is in contact with the subject at the time of being mounted), and the height h1 of the light transmitting section 221 in the intersection point may be used. Similarly, the height of the light transmitting section 221 in the center position F2 of the second light receiver 315 is h2 in FIG. 10A.

Alternatively, when a region including the first light receiver 313 and the light emitting unit 311 is a first region and a region including the second light receiver 315 and the light emitting unit 311 is a second region in a plan view seen from the subject side, the height h1 may be an average height of the light transmitting section 221 in the first region, and the height h2 may be an average height of the light transmitting section 221 in the second region.

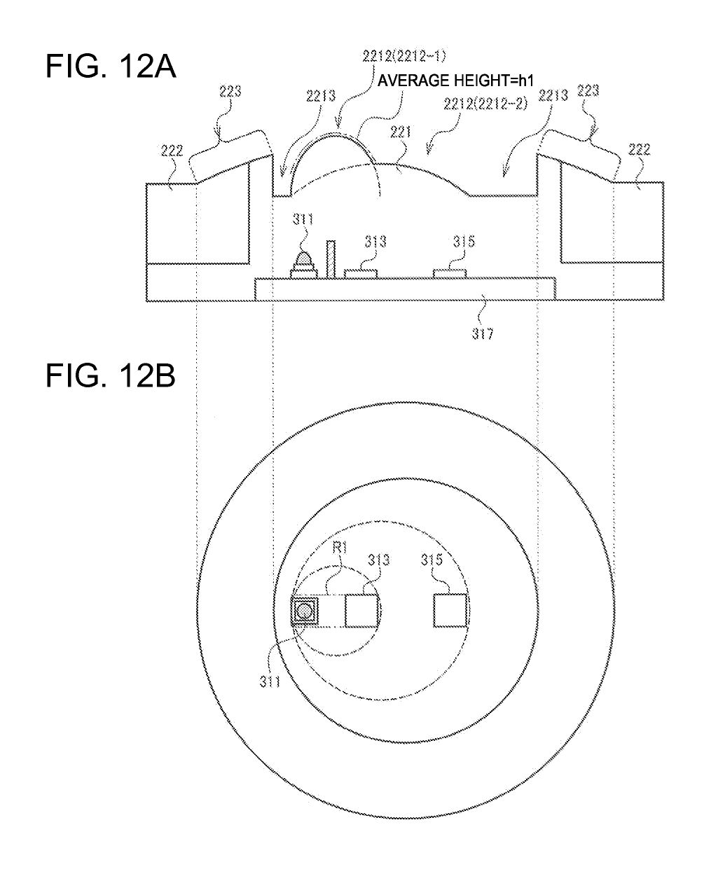

Here, the plan view seen from the subject side indicates a state where a DR3 direction is observed from a point of view which is set closer on the subject side (the DR2 side) than the light emitting unit 311 or the like in FIG. 10A, and specifically, a state of FIG. 10B. In addition, the region including the light emitting unit 311 and the light receiver is also variously considered, and as an example, a region which includes the light emitting unit 311 and the light receiver, and is formed in the shape of a rectangle having the minimum area may be considered. In this case, a region corresponding to the first light receiver 313 (the first region) is R1 in FIG. 12B.

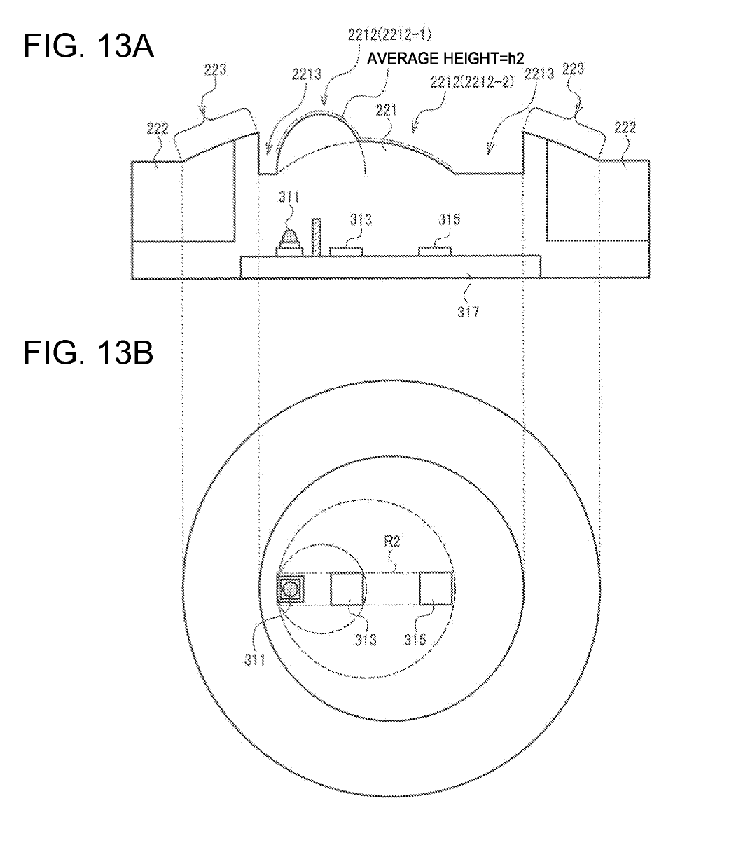

Then, the height of the light transmitting section 221 in the region corresponding to the first light receiver 313 may be defined as an intersection point between a straight line extending in the DR2 direction from each point included in R1 and the surface of the light transmitting section 221, and the height of the light transmitting section 221 in the intersection point may be obtained by being averaged. For example, the average value of the height of the light transmitting section 221 in a region illustrated in FIG. 12A is h1. Furthermore, one sectional surface is illustrated in FIG. 12A, and the height may be averaged in a depth direction of FIG. 12A. Similarly, as illustrated by R2 in FIG. 13B, a region corresponding to the second light receiver 315 (the second region) may be set, and the average height in a range illustrated in FIG. 13A may be h2.

Furthermore, here, an example is described in which the sensor 30 includes two light receivers, and two convex portions 2212 of the light transmitting section 221 are disposed, but the configuration is not limited thereto. For example, the sensor 30 may include one light receiver, and in this case, one convex portion 2212 may be disposed as illustrated in FIG. 1 or the like.

In addition, as described above, the sensor 30 and the convex portion 2212 are described, and a structure other than the convex portion 2212 may be disposed in the detection window 2211 and in the vicinity thereof. Specifically, as illustrated in FIG. 10A or the like, the convex portion 2212 which applies the pressing force to a biological surface of the subject by being in contact with the biological surface, and a groove portion 2213 which is disposed in the vicinity of the convex portion 2212 (to surround the convex portion 2212) may be disposed in the light transmitting section 221 (in a narrow sense, the detection window 2211 of the light transmitting section 221) at the time of mounting the biological information detecting device 1 (at the time of mounting the biological information detecting device 1 on the subject).

Then, in a plan view in which the bottom case 22 is seen from the direction perpendicular to the contact surface with respect to the subject, a pressing force suppressor 223 may be disposed to surround the convex portion 2212 and to suppress the pressing force which is applied to the subject by the convex portion 2212. The pressing force suppressor 223 is disposed in the vicinity of the convex portion 2212 (to surround the convex portion 2212) on a housing surface (a surface on the subject side) of the biological information detecting device 1, and suppresses the pressing force which is applied to the subject by the convex portion 2212.

Then, in this embodiment, the convex portion 2212-1 of the two convex portions 2212 protrudes to the subject side from a pressing force suppressing surface of the pressing force suppressor 223 such that Δh>0 is satisfied. That is, the convex portion 2212-1 protrudes to the subject side from the pressing force suppressing surface of the pressing force suppressor 223 by Δh. This corresponds to h1>h3 in FIG. 10A.

Thus, the convex portion 2212-1 is disposed such that Δh>0 is satisfied, and thus for example, an initial pressing force for exceeding a vein vanishing point is able to be applied to the subject. In addition, the pressing force suppressor 223 for suppressing the pressing force which is applied to the subject by the convex portion 2212-1 is disposed, and thus a variation in the pressing force is able to be suppressed to be minimum in a use range in which the biological information is measured by the biological information detecting device 1, and the noise component or the like is reduced. In addition, when the convex portion 2212-1 protrudes from the pressing force suppressing surface such that Δh>0 is satisfied, the convex portion 2212-1 applies the initial pressing force to the subject by being in contact with the subject, and then the pressing force suppressing surface of the pressing force suppressor 223 is in contact with the subject, and the convex portion 2212-1 is able to suppress the pressing force applied to the subject. Here, the vein vanishing point is a point in which a signal caused by the vein which is superimposed on the pulse wave signal is vanished at the time of gradually increasing the pressing force by bringing the convex portion 2212-1 into contact with the subject, or decreases to the extent of not affecting the measurement of the pulse wave, and corresponds to p2 in FIG. 11.

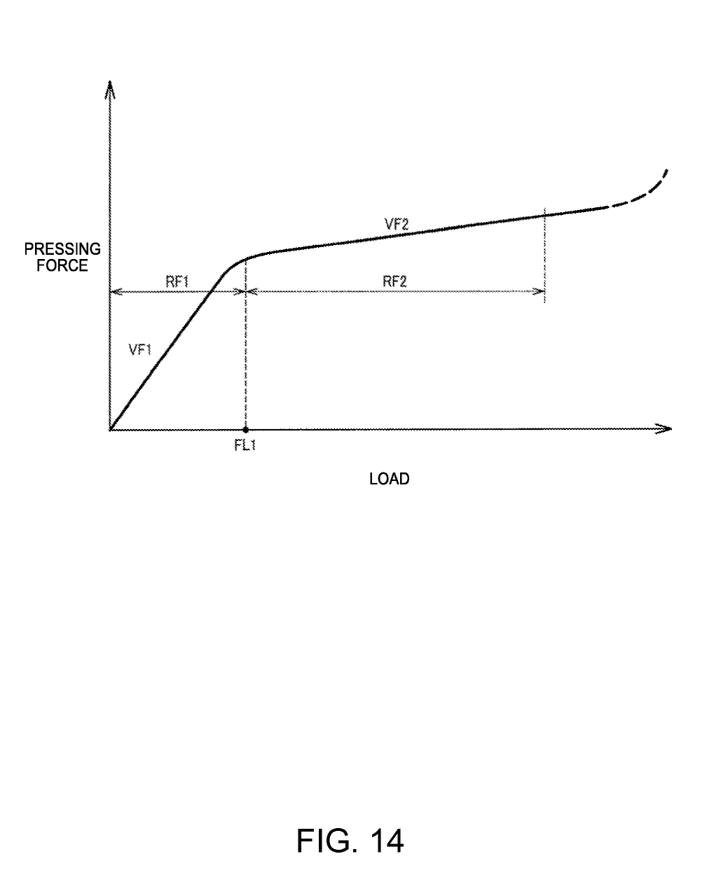

For example, in FIG. 14, a horizontal axis indicates a load generated by a band or the like, and a vertical axis indicates the pressing force applied to the subject by the convex portion 2212-1 (the pressure applied to the blood vessel). Then, a change amount of the pressing force of the convex portion 2212-1 with respect to a load of a load mechanism generating the pressing force of the convex portion 2212-1 is a pressing force change amount. The pressing force change amount corresponds to the inclination of the change properties of the pressing force with respect to the load.

In this case, the pressing force suppressor 223 suppresses the pressing force applied to the subject by the convex portion 2212-1 such that a pressing force change amount VF2 in the second load range RF2 in which the load of the load mechanism is greater than FL1 is smaller than a pressing force change amount VF1 in the first load range RF 1 in which the load of the load mechanism is 0 to FL1. That is, in the first load range RF1 which is an initial pressing force range, the pressing force change amount VF1 increases, and in the second load range RF2 which is the use range of the biological information detecting device 1, the pressing force change amount VF2 decreases.

That is, in first load range RF1, the pressing force change amount VF1 increases, and the inclination of the change properties of the pressing force with respect to the load increases. The pressing force having such a large inclination of the change properties is realized by Δh corresponding to a protruding amount of the convex portion 2212-1. That is, the convex portion 2212-1 is disposed such that Δh>0 is satisfied, and thus even when the load of the load mechanism decreases, it is possible to apply the initial pressing force necessary and sufficient for exceeding the vein vanishing point to the subject.

On the other hand, in the second load range RF2, the pressing force change amount VF2 decreases, and the inclination of the change properties of the pressing force with respect to the load decreases. The pressing force having such a small inclination of the change properties is realized by suppressing the pressing force using the pressing force suppressor 223. That is, the pressing force which is applied to the subject by the convex portion 2212-1 is suppressed by the pressing force suppressor 223, and thus even when there is a variation in the load or the like in the use range of the biological information detecting device 1, it is possible to suppress the variation in the pressing force to be minimum. Accordingly, the noise component or the like is reduced.

Thus, the optimized pressing force (for example, approximately 16 kPa) is applied to the subject, and thus it is possible to obtain a pulse wave detection signal having a higher M/N ratio (S/N ratio). That is, it is possible to increase the signal component of the pulse wave sensor 31 and to reduce the noise component. Here, M indicates a signal level of the pulse wave detection signal, and N indicates a noise level.

In addition, the height of a bottom surface of the groove portion 2213 is lower than the height of an outer surface (a surface on the subject side at the time of being mounted) of the pressing force suppressor 223 (the height in the highest end portion), and the bottom surface of the groove portion 2213 is a surface which is lower than the outer surface (on the sensor 30 side).

For example, when a relatively soft material such as the skin is in contact with a contact surface of the light transmitting section 221 of a hard material formed of a resin, a region which is not in contact with the skin or a region having a weak contact force is generated in the vicinity of a circumferential edge portion (an outer circumferential portion) of the light transmitting section 221. Accordingly, for example, when a flat portion is formed around the convex portion 2212 without disposing the groove portion 2213, the flat portion is not in contact with the skin or a weak contact state occurs, and thus the contact state is dynamically changed. Then, due to the dynamic change in the contact state, the strength and weakness of the light easily optically occurs, and when such light is incident on the light receiver, the light becomes a noise which is not relevant to the pulse component.

From this point, when the groove portion 2213 as illustrated in FIG. 10A is disposed, it is possible to effectively prevent a region in which the contact state is dynamically changed from being generated, and thus the signal integrity or the like is improved.

### 4. Modification Example

In this embodiment as described above, the light shielding section 222 is formed of the glass-containing resin material. However, the object of this embodiment is to realize the biological information detecting device 1 having a reduced size and weight while increasing the waterproof properties, and it may not be necessary that the light shielding section 222 is formed of the glass-containing resin material.

Specifically, the biological information detecting device 1 according to this embodiment may include the sensor 30 detecting the biological information of the subject, and the case 20 in which the sensor 30 is housed (disposed), the case 20 may include the top case 21 and the bottom case 22, the bottom case 22 may include the light transmitting section 221 including the detection window 2211 through which the light incident on the sensor 30 is transmitted, and the light shielding section 222 disposed in the vicinity of the light transmitting section 221, and the light transmitting section 221 may extend from the detection window 2211 to the sealing portion 50 which is disposed in the connection portion between the top case 21 and the bottom case 22.

Specifically, the biological information detecting device 1 according to this embodiment has the structure illustrated in FIG. 1. An advantage of forming the light transmitting section 221 to extend is described above, and the packing 52 used in the waterproofing of the connection portion between the top case 21 and the bottom case 22 is also used in the waterproofing between the light transmitting section 221 and the light shielding section 222, and thus it is possible to increase the waterproof properties by a simple configuration. That is, even when the light shielding section 222 is not formed of the glass-containing resin material, it is possible to increase the waterproof properties and to reduce the thickness (suppress an increase in the thickness by increasing the waterproof properties), and thus a structure solving the problems described above is obtained. Further, from a viewpoint of emphasizing the waterproof properties and the reduction in the thickness, it is not necessary that the light transmitting section 221 and the light shielding section 222 are integrally formed, and may be separately configured. Even in this case, a waterproof effect is obtained by the packing 52.

In addition, various modification examples are considered in this embodiment such that the light transmitting section 221 is formed to extend, and the light shielding section 222 is formed of the glass-containing resin material, and then the light transmitting section 221 and the light shielding section 222 are separately formed.

Furthermore, as described above, this embodiment is described in detail, but a person skilled in the art is able to easily understand that many modifications are able to be performed without substantially deviating from the new matters and the effects of the disclosure. Accordingly, all of these modification examples are included in the range of the disclosure. For example, in the specification and the drawings, terms described at least once along with different terms having wider meaning or the same meaning are able to be substituted with the different terms in any portion of the specification or the drawings. In addition, the configuration and the operation of the biological information detecting device are not limited to those described in this embodiment, and are able to be variously modified.

What is claimed is:

1. A biological information detecting device, comprising:
a sensor configured to detect biological information of a subject; and
a case housing the sensor, the case includes
a light transmitting section formed of a resin material, the light transmitting section including a detection window through which light incident on the sensor is transmitted, and
a light shielding section formed of a glass-containing resin material, the light shielding section arranged in the vicinity of the light transmitting section,
wherein
the light transmitting section and the light shielding section are integrally formed.

2. The biological information detecting device according to claim 1,
wherein the light transmitting section and the light shielding section are integrally formed by two-color molding or insert molding.

3. The biological information detecting device according to claim 1,
wherein the light shielding section is arranged to be superimposed on the light transmitting section from the subject side of the light transmitting section in a portion other than the detection window.

4. The biological information detecting device according to claim 1,
wherein the case includes
a top case, and
a bottom case including the light transmitting section and the light shielding section, and
the light transmitting section extends from the detection window to a sealing portion which is disposed in a connection portion between the top case and the bottom case.

5. The biological information detecting device according to claim 4,
wherein the sealing portion includes a packing which seals the case.

6. The biological information detecting device according to claim 4,
wherein the light transmitting section is arranged in a region between the detection window and the sealing portion in a plan view in which the bottom case is seen from a direction perpendicular to a contact surface with respect to the subject.

7. The biological information detecting device according to claim 1,
wherein the light transmitting section contains at least one of a polycarbonate, an ABS resin, and an acrylic resin, and
the light shielding section contains at least one of the polycarbonate containing glass, the ABS resin containing glass, and the acrylic resin containing glass.

8. The biological information detecting device according to claim 1,
wherein the light transmitting section, further includes
a convex portion configured to apply a pressing force to a biological surface of the subject by being in contact with the biological surface when being mounted on the subject, and
a groove portion disposed in the vicinity of the convex portion.

9. The biological information detecting device according to claim 8, further comprising:
a pressing force suppressor which is arranged in the vicinity of the convex portion in a plan view in which the case is seen from a direction perpendicular to a contact surface with respect to the subject, and configured to suppress the pressing force applied to the subject by the convex portion.

10. The biological information detecting device according to claim 1,
wherein the sensor includes
a first light receiver and a second light receiver configured to receive light from the subject.

11. The biological information detecting device according to claim 10,
wherein in a direction from the sensor to the subject, when a height of a position or a region of the light transmitting section corresponding to the first light receiver is h1, and a height of a position or a region of the light transmitting section corresponding to the second light receiver is h2, h1>h2 is satisfied.

12. The biological information detecting device according to claim 1, further comprising:
a secondary battery housed in the case; and
a circuit board electrically connected to the sensor,
wherein the secondary battery is arranged between the circuit board and the sensor.

13. The biological information detecting device according to claim 12,
wherein a vibrator is arranged between the secondary battery and the case in a plan view in which the case is seen from a direction perpendicular to a contact surface with respect to the subject.

14. A biological information detecting device, comprising:
a sensor detecting biological information of a subject; and
a case housing the sensor,
wherein the case, includes
a top case, and
a bottom case,
the bottom case, includes
a light transmitting section including a detection window through which light incident on the sensor is transmitted, and
a light shielding section arranged in the vicinity of the light transmitting section, and
the light transmitting section extends from the detection window to a sealing portion which is arranged in a connection portion between the top case and the bottom case.

15. The biological information detecting device according to claim 14,
wherein the light transmitting section is formed of a resin material, and
the light shielding section is formed of a glass-containing resin material in which glass is contained in a resin material.
